(19) United States
(12) Patent Application Publication
(10) Pub. No.: US 2015/0218648 A1
(43) Pub. Date: Aug. 6, 2015

(54) METHODS AND COMPOSITIONS FOR DIAGNOSIS AND PROGNOSIS IN BREAST CANCER

(71) Applicant: PACYLEX PHARMACEUTICALS INC., Edmonton (CA)

(72) Inventors: Luc Gerard Berthiaume, Edmonton (CA); Ryan John Heit, Edmonton (CA)

(21) Appl. No.: 14/418,939

(22) PCT Filed: Aug. 1, 2013
(86) PCT No.: PCT/IB2013/002103

§ 371 Date: Jan. 31, 2015

Related U.S. Application Data
(60) Provisional application No. 61/678,232, filed on Aug. 1, 2012.

(51) Int. Cl. C12Q 1/68; G01N 33/574

(52) U.S. Cl. CPC C12Q 1/6886; G01N 33/57415; C12Q 2600/118; C12Q 2600/16; C12Q 2600/158; G01N 2333/91051

(57) ABSTRACT

The present invention relates to methods for diagnosing breast cancer; methods for assigning risk of reoccurrence of breast cancer; methods for assigning risk of mortality due to breast cancer, methods of monitoring breast cancer; methods of staging breast cancer; and various devices and kits adapted to perform such methods. These methods comprise measurement gene expression from one or more protein fatty acyl transferase genes in a tissue sample, and preferably in a tumor sample, from the patient.

The present application claims priority to U.S. Provisional Patent Application 61/678,232, filed Aug. 1, 2012, which is hereby incorporated in its entirety including all tables, figures, and claims.

### FIELD OF THE INVENTION

The present invention relates to methods and compositions for diagnosis, prognosis, and monitoring breast cancer.

### BACKGROUND OF THE INVENTION

The following discussion of the background of the invention is merely provided to aid the reader in understanding the invention and is not admitted to describe or constitute prior art to the present invention.

Breast cancer is the most common invasive cancer in women (accounting for about 16% of all female cancers and approximately 7.6 million deaths worldwide in 2008), and the second most common cause of cancer death worldwide after lung cancer. It is less common in men and in developing countries. Approximately ten to twenty percent of cases in the United States exhibit a genetic component. BRCA1 and BRCA2 are among the gene mutations known to contribute to this genetic component. Mutations in these genes confer a startlingly large lifetime risk of breast cancer—between 60 and 85 percent, and the likelihood that breast cancer is genetically associated is highest in families with a history of multiple cases of breast and ovarian cancer. Other risk factors that increase the likelihood of developing breast cancer include smoking, consumption of alcohol, high fat intake, and obesity.

Breast cancers are classified by several grading systems. These include histological appearance, grade, stage, and receptor status. Staging under the current TNM system is based on the size of the tumor (T), whether or not the tumor has spread to the lymph nodes (N) in the armpits, and whether the tumor has metastasized (M). Stage 0 is a pre-cancerous or marker condition, either ductal carcinoma in situ (DCIS) or lobular carcinoma in situ (LCIS). Stages 1-3 refer to cancers that remain within the breast or regional lymph nodes. Stage 4 refers to metastatic disease.

In terms of receptor status, the most important currently are estrogen receptor (ER), progesterone receptor (PR), and HER2. Receptor status can be used to guide certain treatment choices. ER+ cancer cells depend on estrogen for their growth, so they can be treated with drugs to block estrogen effects (e.g. tamoxifen). HER2+ breast cancer can be treated with drugs such as the monoclonal antibody trastuzumab (Herceptin®) targeting the HER2 molecule. Cells with none of these receptors are termed “triple negative”. Triple-negative breast cancer accounts for approximately 15%-25% of all breast cancer cases, and have an increased risk of relapse in the near term (3-5 years) but this risk drops below that of hormone-positive breast cancers at later time points.

Expression of estrogen receptor is currently the most clinically useful test to predict hormone responsiveness of breast cancer. Nevertheless, thirty percent of ER-positive breast cancers do not respond to anti-estrogens such as Tamoxifen. Similarly, a substantial number of patients with HER2 amplified breast cancer experience recurrence or progression which is ultimately fatal. These treatments are not without serious side effects. In the case of Herceptin, the drug cost for 52 weeks of therapy in the United States is approximately $100,000, and approximately 5% to 15% of patients develop cardiac dysfunction while taking Herceptin.

There remains a need in the art for markers which can be used for diagnosis and risk stratification of patients having or suspected of having breast cancer, and for assessment of therapeutic efficacy in patients receiving therapy for breast cancer.

### BRIEF SUMMARY OF THE INVENTION

It is an object of the invention to provide methods and compositions for diagnosis, prognosis, and determination of treatment regimens in subjects suffering from, or being evaluated for, breast cancer. In various aspects, the present invention provides methods for diagnosing breast cancer; methods for assigning risk of reoccurrence of breast cancer; methods for assigning risk of mortality due to breast cancer, methods of monitoring breast cancer; methods of staging breast cancer; and various devices and kits adapted to perform such methods.

In a first aspect, the present invention relates to methods for determining a prognostic classification for a patient diagnosed with breast cancer. These methods comprise (i) obtaining a tumor cell sample from the patient; (ii) generating one or more assay results indicative of the gene expression of one or more, and preferably a plurality, of protein fatty acyl transferase genes in the tumor cell sample; and (iii) calculating the prognostic classification by correlating the assay results obtained to a likelihood of an outcome result for the patient.

In various embodiments, the likelihood of an outcome result is a likelihood that the patient will respond to a selected treatment regimen; a likelihood of tumor recurrence; a likelihood of metastatic disease; and/or a likelihood of mortality. The likelihood of an outcome result may be expressed in a variety of forms known to those of skill in the art for expression of a relative or absolute risk, such as an odds ratio, a hazard ratio, a risk ratio, a quantile rank, a time-to-event, a cumulative risk, an incidence rate, etc.

In certain embodiments, the one or more (or plurality) of protein fatty acyl transferase genes comprise one or more of genes selected from the group consisting of NMT1, NMT2, Hhat, Pore, zDHHC-3, zDHHC-5, zDHHC-7, zDHHC-9, zDHHC-14, zDHHC-20, zDHHC-23 and zDHHC-24. In preferred embodiments, the plurality of protein fatty acyl transferase genes comprises one, two, three, or more of zDHHC-5, zDHHC-7, zDHHC-9, zDHHC-14 and zDHHC-20.

The one or more assay results used in calculating the prognostic classification may be individual assay results, each of which is indicative of the gene expression level of one protein fatty acyl transferase gene; or the assay results may be expressed as a single value that is a function of the plurality of individual assay results.

The prognostic classification may be based solely on the protein fatty acyl transferase gene expression data, or may optionally further include one or more assay results indicative of the expression of one or more biomarkers known to be related to prognostic classification for a patient diagnosed with breast cancer. Examples of such genes include, but are not limited to, H-Ras, N-Ras, estrogen receptor, progesterone receptor, HER1, HER2, Ki67 and cytokeratin 5/6.

Various methods may be used to generate the one or more assay results indicative of the gene expression of the plurality of protein fatty acyl transferases genes, including the detection of either RNA or protein expressed from the genes of interest.

In the case of RNA detection, a set of amplified nucleic acids may be prepared by enzymatically amplifying mRNA sequences present in an RNA sample using a primer-dependent nucleic acid polymerase, wherein primers used to prepare the set of amplified nucleic acids are selected to amplify mRNA expressed by the plurality of protein fatty acyl transferase genes. The one or more assay results may be generated from the set of amplified nucleic acids by various hybridization or sequencing techniques, including quantitative reverse transcription-polymerase chain reaction (qRT-PCR), as described hereinafter. In addition, direct detection of target mRNA may be performed without an amplification step. Multiparameter or multiplexed detection and quantification methods that simultaneously measures multiple and different mRNA species are disclosed, for example, in US2007/0166708 and US20070292877, which are hereby incorporated by reference in their entirety.

In the case of protein detection, the one or more assay results may be generated by contacting a protein sample with a plurality of reagents which specifically bind for detection proteins expressed from the plurality of protein fatty acyl transferase genes, and generating one or more assay results indicative of the gene expression of the plurality of protein fatty acyl transferase genes from protein bound to the plurality of reagents. Typically, the specific binding reagents are based on an immunoglobulin scaffold (e.g., antibodies), but other specific binding reagents such as isolated receptors, aptamers, etc., may be employed. For simplicity, specific binding of target proteins will be referred to herein as “immunoassay detection” of proteins, regardless of the choice of specific binding reagent which is being employed Immunoassay detection may be performed in a variety of formats, including, but not limited to, antibody microarray, Western blot, SELDI-TOF-MS, fluorescent immunoassay, ELISA, or flow cytometry.

As noted, in certain embodiments described herein, the assay result(s) obtained is(are) compared to a corresponding baseline (i.e., a diagnostic or prognostic “threshold”) level which is considered indicative of a “positive” or “negative” result. A variety of methods may be used by the skilled artisan to arrive at a desired baseline. In certain preferred embodiments, the baseline assay result is determined from an earlier assay result obtained from the same subject. That is, the change in a biomarker concentration may be observed over time, and an increased concentration provides an indication of breast cancer in the subject. In alternative embodiments, the baseline assay result is determined from a population of subjects. In the case of the use of the markers of the present invention for diagnosis, the population may contain some subjects which suffer from breast cancer, and some which do not; in the case of their use for prognosis, the population may contain some subjects which suffer from some outcome (e.g., recurrence of breast cancer; metastasis of breast cancer; worsening stage of breast cancer, etc.), and some which do not. As described hereinafter, a threshold is selected which provides an acceptable level of specificity and sensitivity in separating the population into a “first” subpopulation exhibiting a particular characteristic (e.g., having breast cancer) relative to the remaining “second” subpopulation that does not exhibit the characteristic. As discussed herein, a preferred threshold value separates this first and second population by one or more of the following measures of test accuracy:

an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less.
at least 75% sensitivity, combined with at least 75% specificity;
a Receiver Operating Characteristic (ROC) curve area of at least 0.6, more preferably 0.7, still more preferably at least 0.8, even more preferably at least 0.9, and most preferably at least 0.95; and/or
a positive likelihood ratio (calculated as sensitivity/(1-specificity)) of at least 5, more preferably at least 10, and most preferably at least 20; or
a negative likelihood ratio (calculated as (1-sensitivity)/specificity) of less than or equal to 0.3, more preferably less than or equal to 0.2, and most preferably less than or equal to 0.1. The term “about” in this context refers to +/−5% of a given measurement.

In certain embodiments, the methods of the present invention further comprise selecting a treatment regimen based on the prognostic classification. Once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17th Ed. Merck Research Laboratories, Whitehouse Station, N.J., 1999. Since the methods and compositions described herein provide prognostic information, the markers of the present invention may be used to initiate and monitor a course of treatment. For example, improved or worsened prognostic state may indicate that a particular treatment is or is not efficacious.

In various related aspects, the present invention relates to devices and kits for performing the methods described herein. Suitable kits comprise reagents sufficient for performing at least one of the described assays, together with instructions for performing the described baseline comparisons. Such kits can optionally include comparable reagents and instructions for measuring and using additional variables such as H-Ras, N-Ras, estrogen receptor, progesterone receptor, HER1, HER2, Ki67, and cytokeratin 5/6, etc., as described above.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE FIGURES

FIG. 1 depicts a “heat map” showing the averaged expression (log₂ transformed) of various PFATs per tumor subtype, and the average expression of each PFAT plotted as a Z score distance from the mean of the normal breast tissue.

FIG. 2 depicts a combinatorial prognostic analysis of zDHHC-PATs-7, -14 and -20 expression.

### DETAILED DESCRIPTION OF THE INVENTION

The present invention relates to methods and compositions for diagnosis, prognosis, and determination of treatment regimens in subjects suffering from breast cancer.

Breast cancer is the leading cause of death from cancer in women in North America and has an approximate 20% mortality rate. Carcinoma of the breast will affect 1 in 8 to one in 12 women over their life-time. Breast cancer is also a leading cause of death worldwide and accounts for approximately 0.9% of annual global deaths.

Breast cancer is a heterogeneous disease with respect to sensitivity to treatment, clinical outcomes, genetic alterations, and molecular classification; certain subtypes consistently carry poorer prognoses. This diversity poses a challenge in developing tumor classifications that can be used to individualize risk assessment and treatment decisions. One widely accepted molecular classification of invasive breast cancer relies on the presence or absence of protein markers defined as: Luminal A (estrogen receptor [ER] and/or progesterone receptor [PR] positive [+] and human epidermal growth factor receptor-2 negative [HER2−]), Luminal B (ER+ and/or PR+, HER2+), Basal-like (ER−, PR−, HER2−, cytokeratin 5/6+ and/or HER1+), HER2+/ER−(ER−, PR−, HER2+) and unclassified (negative for all 5 biomarkers). Of these 5 subtypes, the poorest prognosis occurs in the basal-like, unclassified and HER2+/ER− groups, the combination of which account for 35% of breast cancers. Such validated prognostic and predictive markers contribute important information to guide breast cancer treatment decisions. For example, the therapeutic monoclonal anti-Her2 antibody Trastuzumab is used exclusively in the treatment of patients with HER2+ subtypes and its application has markedly improved outcomes in both the adjuvant (curative intent) setting and in the palliation of metastatic breast cancer.

Because of its role in membrane binding, protein fatty acylation plays a critical role in signal transduction, a process often altered in cancer cells. Examples of fatty acylated proteins linked to cancer include Src, Abl, Ras, Hedgehog and Wnt oncoproteins. The enzymes that modify proteins with fatty acids are protein fatty acyl transferases. Because several oncoproteins are palmitoylated variation in the expression of protein fatty acyl transferases may determine the signal transducing properties of oncogenic proteins and the cellular complement of fatty acylated proteins in poor-prognosis breast cancers. The present invention demonstrates that over-expression of fatty acyl transferase genes, and in particular a subset of transferases named zDHHC-5, -7, -9, -14 and -20, are highly correlated to the rate of tumour recurrence and death. Furthermore, the patients whose biopsies showed normal levels of zDHHC-7, -14 and -20 in comparison to normal mastectomies had a substantially better survival rate than those with elevated levels of these markers.

The terms “marker” and “biomarker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “The molecules used as markers in the present invention are contemplated to include any fragments of a parent biomarker, in particular, immunologically detectable fragments of protein biomarkers and specifically hybridizable lengths of nucleic acids such as mRNAs. Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result.

As used herein, the term “relating a signal to the presence or amount” of an analyte reflects this understanding. Assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Because an antibody epitope is on the order of 8 amino acids, an immunoassay configured to detect a marker of interest will also detect polypeptides related to the marker sequence, so long as those polypeptides contain the epitope(s) necessary to bind to the antibody or antibodies used in the assay. In the case of nucleic acids, an assay will be configured to detect a marker of interest so long as it has the sequences necessary for detection (e.g., by hybridization) and, if necessary, amplification of the target of interest.

In this regard, the skilled artisan will understand that the signals obtained from an immunoassay or a nucleic acid hybridization are a direct result of complexes formed between one or more labels and the target biomolecule (i.e., the analyte). While such assays may detect the full length biomarker and the assay result be expressed as a concentration of a biomarker of interest, the signal from the assay is actually a result of all such “immunoreactive” polypeptides or “hybridizable” nucleic acids present in the sample.

Preferred assays are “configured to detect” a particular marker. That an assay is “configured to detect” a marker means that an assay can generate a detectable signal indicative of the presence or amount of a physiologically relevant concentration of a particular marker of interest. Such an assay may, but need not, specifically detect a particular marker

The term “test sample” as used herein refers to a biological sample obtained from a subject for the purpose of diagnosis, prognosis, or evaluation of the subject of interest, such as a patient. In certain embodiments, such a sample may be obtained for the purpose of determining the outcome of an ongoing condition or the effect of a treatment regimen on a condition. Preferred test samples include tissue, blood, serum, plasma, cerebrospinal fluid, urine, saliva, sputum, breast juices, and pleural effusions. In addition, one of skill in the art would realize that some test samples would be more readily analyzed following a fractionation or purification procedure, for example, separation of whole blood into serum or plasma components or extraction of nucleic acids. Any suitable methods for obtaining a biological sample can be employed, e.g., a biopsy procedure, a fine needle aspiration, an excision of tissue, etc. A sample may be processed in any suitable manner after being obtained from the individual. For example, preparation of a tissue sample for histological evaluation involves fixation, dehydration, infiltration, embedding, and sectioning.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care for a disease or condition. This includes persons with no defined illness who are being investigated for signs of pathology.

Use of Biomarkers in Medicine

There is substantial interest in determining gene expression patterns that can provide prognostic data, particularly in cancer recurrence and for managing cancer treatment. As opposed to genetic tests, which measure an individual's inherited genetic makeup and are utilized to estimate the risk of developing a disease (e.g., cancer), such gene-profiling assay measures the expression of genes associated with an individual's tumor. Patterns of gene expression can be compared to outcome databases to identify specific patterns that may be associated with prognosis. It is believed this information may then be used to predict an individual's likelihood of cancer recurrence, and in the clinical decision-making process regarding the use of adjuvant chemotherapy and/or optional chemotherapy regimen. Examples of these assays include: Oncotype DX™ (21-gene panel); MammaPrint® (70-gene panel; also referred to as the “Amsterdam signature”); Mammostrat™ (Applied Genomics Inc.), the Molecular Grade Index (Aviara MGISM, AviaraDX, Inc.), THEROS Breast Cancer IndexSM, BreastOncPx, NexCourse Breast IHC4, and the PAM50 Breast Cancer Intrinsic Classifier.

The present invention demonstrates that protein fatty acyl transferase genes such as NMT1, NMT2, Hhat, Porc, zDHHC-3, zDHHC-5, zDHHC-7, zDHHC-9, zDHHC-14, zDHHC-20, zDHHC-23 and zDHHC-24 can provide prognostic data, particularly in breast cancer patients. These gene(s) can be used by themselves, or can be combined with previously known genes or expression panels to assign a likelihood of an outcome result for the patient (e.g., a likelihood of recurrence, a likelihood of survival, etc.) and to guide therapeutic decisions. By way of example, gene expression of one or more protein fatty acyl transferase genes may be combined with one or more biomarkers selected from the group consisting of H-Ras, N-Ras, estrogen receptor, progesterone receptor, HER1, HER2, and cytokeratin 5/6; and/or with one or more gene expression profiles indicative of breast cancer prognosis such as a PAM50 risk of recurrence score, a Breast Cancer Index score, a DCIS Score, an Oncotype DX recurrence score, an IHC4 recurrence risk score, etc. This list is not meant to be limiting.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition. The term “diagnosis” does not refer to the ability to determine the presence or absence of a particular disease with 100% accuracy, or even that a given course or outcome is more likely to occur than not. Instead, the skilled artisan will understand that the term “diagnosis” refers to an increased probability that a certain disease is present in the subject.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing morbidity or mortality in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient.

A biomarker that is either over-expressed or under-expressed can also be referred to as being “differentially expressed” or as having a “differential level” or “differential value” as compared to a “non-diseased” expression level or value of the biomarker that indicates or is a sign of a normal process or an absence of a disease or other condition in an individual. Thus, “differential expression” of a biomarker can also be referred to as a variation from a “non-diseased” expression level of the biomarker.

The term “differential gene expression” and “differential expression” are used interchangeably herein to refer to a marker (e.g., a RNA or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide.

Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

The term “correlating” or “relating” as used herein in reference to the use of markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome using ROC curves.

In certain embodiments, the methods described herein comprise the comparison of an assay result to a corresponding baseline result. The term “baseline result” as used herein refers to an assay value that is used as a comparison value (that is, to which a test result is compared). In practical terms, this means that a marker is measured in a sample from a subject, and the result is compared to the baseline result. A value above the baseline indicates a first likelihood of a diagnosis or prognosis, and a value below the baseline indicates a second likelihood of a diagnosis or prognosis.

A baseline can be selected in a number of manners well known to those of skill in the art. For example, data for a marker or markers (e.g., concentration in a body fluid, such as urine, blood, serum, breast juices, biopsies, or plasma) may be obtained from a population of subjects. The population of subjects is divided into at least two subpopulations. The first subpopulation includes those subjects who have been confirmed as having a disease, outcome, or, more generally, being in a first condition state. For example, this first subpopulation of patients may be those diagnosed with breast cancer, and that suffered from a worsening of cancer or suffered from mortality as a result of their cancer. For convenience, subjects in this first subpopulation will be referred to as “diseased,” although in fact, this subpopulation is actually selected for the presence of a particular characteristic of interest. The second subpopulation of subjects is formed from the subjects that do not fall within the first subpopulation. Subjects in this second set will hereinafter be referred to as “non-diseased.”

A baseline result may then be selected to distinguish between the diseased and non-diseased subpopulation with an acceptable specificity and sensitivity. Changing the baseline merely trades off between the number of false positives and the number of false negatives resulting from the use of the particular marker under study. The effectiveness of a test having such an overlap is often expressed using a ROC curve. ROC curves are well known to those skilled in the art. The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In an alternative, an individual subject may provide their own baseline, in that a temporal change is used to indicate a particular diagnosis or prognosis. For example, one or more markers may be determined at an initial time to provide one or more baseline results, and then again at a later time, and the change (or lack thereof) in the marker level(s) over time determined. In such embodiments, an increase in the marker from the initial time to the second time may be indicative of a particular prognosis, of a particular diagnosis, etc. Likewise, a decrease in the marker from the initial time to the second time may be indicative of a particular prognosis, of a particular diagnosis, etc. In such an embodiment, a plurality of markers need not change in concert with one another. Temporal changes in one or more markers may also be used together with single time point marker levels compared to a population-based baseline.

In certain embodiments, a baseline marker level is established for a subject, and a subsequent assay result for the same marker is determined That subsequent result is compared to the baseline result, and a value above the baseline indicates worsening cardiac function, relative to a value below the baseline. Similarly, a value below the baseline indicates improved cardiac function, relative to a value above the baseline.

In certain embodiments, a baseline marker level is established for a subject, and a subsequent assay result for the same marker is determined That subsequent result is compared to the baseline result, and a value above the baseline indicates an increased mortality risk, relative to a value below the baseline. Similarly, a value below the baseline indicates a decreased mortality risk, relative to a value above the baseline.

As discussed herein, the measurement of the level of a single marker may be augmented by additional markers. Various clinical variables may also be utilized as variables in the methods described herein. Examples of such variables include stage of the breast cancer identified histologically, smoking status, etc. This list is not meant to be limiting.

Suitable methods for combining markers into a single composite value that may be used as if it is a single marker are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims. In an alternative, assay results may be used in an “n-of-m” type of approach. Using a two marker example of such methods, when either marker above its corresponding baseline value may signal a breast cancer diagnosis or an increased risk of an adverse outcome (in n-of-m terms, this is a “1-of-2” result). If both are above the corresponding baselines (a “2-of-2” result), an even greater confidence in the subject's status may be indicated.

The sensitivity and specificity of a diagnostic and/or prognostic test depends on more than just the analytical “quality” of the test—they also depend on the definition of what constitutes an abnormal result. In practice, ROC curves, are typically calculated by plotting the value of a variable versus its relative frequency in “normal” and “disease” populations. For any particular marker, a distribution of marker levels for subjects with and without a “disease” will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

Measures of test accuracy may also be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures.

Preferably, a baseline is chosen to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

Assays Applicable to Protein Biomarkers

In the case of measuring protein expression of a biomarker, preferably such markers are analyzed using an immunoassay, and most preferably sandwich immunoassay, although other methods are well known to those skilled in the art. The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Biological assays such as immunoassays require methods for detection, and one of the most common methods for quantitation of results is to conjugate an enzyme, fluorophore or other molecule to form an antibody-label conjugate. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). Particularly preferred detectable labels are fluorescent latex particles such as those described in U.S. Pat. Nos. 5,763,189, 6,238,931, and 6,251,687; and International Publication WO95/08772, each of which is hereby incorporated by reference in its entirety. Exemplary conjugation to such particles is described hereinafter. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The term “solid phase” as used herein refers to a wide variety of materials including solids, semi-solids, gels, films, membranes, meshes, felts, composites, particles, papers and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Dianiandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997, 1998; Kessler et al., Agnew. Chem. Int. Ed. 40: 165, 2001; Smith et al., J. Comb. Med. 1: 326, 1999; Orain et al., Tetrahedron Lett. 42: 515, 2001; Papanikos et al., J. Am. Chem. Soc. 123: 2176, 2001; Gottschling et al., Bioorg. Med. Chem. Lett. 11: 2997, 2001. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot. When multiple assays are being performed, a plurality of separately addressable locations, each corresponding to a different marker and comprising antibodies that bind the appropriate marker, can be provided on a single solid support. The term “discrete” as used herein refers to areas of a surface that are non-contiguous. That is, two areas are discrete from one another if a border that is not part of either area completely surrounds each of the two areas. The term “independently addressable” as used herein refers to discrete areas of a surface from which a specific signal may be obtained.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of markers using a single test device. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody conjugated to a solid phase and a second antibody conjugated to a signal development element. Such assay devices are configured to perform a sandwich immunoassay for one or more analytes. Other preferred methods of specifically detecting protein biomarkers include, but are not limited to, dot blots, western blots, chromatographic methods, mass spectrometry (e.g., SELDI-TOF), and flow cytometry-based methods such as xMAP® (Luminex Corporation).

Flow of a sample in an assay device along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001, which is hereby incorporated by reference in its entirety.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay(s) of interest. Optionally the kits may contain one or more means for using information obtained from immunoassays or other specific binding assays performed for a marker panel to rule in or out certain diagnoses or prognoses. Other measurement strategies applicable to the methods described herein include chromatography (e.g., HPLC), mass spectrometry, receptor-based assays, and combinations of the foregoing.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.” While the present invention is described in detail in terms of immunologic detection of an analyte, other marker binding partners such as aptamers, receptors, binding proteins, etc., may be used in a similar fashion to antibodies in providing an assay.

Preferably, an antibody or other binding partner used in an assay is selected that specifically binds a marker of interest. The term “specifically binds” is not intended to indicate that an antibody/binding partner binds exclusively to its intended target. Rather, an antibody/binding partner “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10″ M⁻¹.

Affinity is calculated as Kₐ=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):
where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Since the discovery of aptamers, many researchers have used aptamer selection as a means for generation of suitable binding partners for binding assay.

Those skilled in the art will recognize that many approaches can be taken in producing antibodies or other binding partners, and screening and selecting for affinity and specificity for use in biomarker assays, but these approaches do not change the scope of the invention.

Assays Applicable to Nucleic Acid Biomarkers

The term “gene expression profiling” is used herein in the broadest sense, and includes methods of quantification of mRNA and/or protein levels in a biological sample. Thus, measuring gene expression by detection of RNA in a biological sample may be used as a surrogate for detection of the level of the corresponding protein in the biological sample. Thus, any of the biomarkers or biomarker panels described herein can also be detected by detecting the appropriate RNA.

Northern blots, microarrays, Invader assays, and RT-PCR combined with capillary electrophoresis have all been used to measure expression levels of mRNA in a sample. mRNA expression levels may also be measured by reverse transcription quantitative polymerase chain reaction (RT-PCR followed with qPCR). In these methods, RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. See Gene Expression Profiling: Methods and Protocols, Richard A. Shimkets, editor, Humana Press, 2004.

The first analytical step in a nucleic acid-based gene expression profiling method is the extraction and purification of RNA to be analyzed from biological samples. The starting material can, for example, be total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, head and neck, etc., tumor, or tumor cell lines. If the source of mRNA is a primary tumor, mRNA can be extracted, for example, from frozen or archived paraffin-embedded fixed (e.g. formalin-fixed) tissue samples (FPE tissues). If the RNA source is from FPE tissues, this method includes the removal of paraffin. It is well known that deparaffinization of FPE tissues can be accomplished by protocols employing xylenes as a solvent. Alternatively, RNA can be extracted and purified using a protocol in which dewaxing is performed without the use of any organic solvent, thereby eliminating the need for multiple manipulations associated with the removal of the organic solvent, and substantially reducing the total time to the protocol. According to this alternative protocol, wax, e.g. paraffin, is removed from wax-embedded tissue samples by incubation at 65-75° C. in a lysis buffer that solubilizes the tissue and hydrolyzes the protein, followed by cooling to solidify the wax. After extraction, the RNA is then incubated with DNase 1 by standard methods to ensure that DNA contamination of the purified RNA is kept below a threshold above which the presence of genomic DNA would compromise accurate qRT-PCR measurement of mRNA species.

One method of accomplishing quantitative recovery of purified nucleic acids is to use carrier-mediated precipitation of the purified material. Alternatively, chromatographic or affinity capture and release based methods may be used to recover selective fractions of the purified nucleic acid. These methods may include a variety of membranes or matrices with size exclusion properties or affinity membranes or matrices requiring prior modification of the purified nucleic acid with a hapten or “capture nucleotide sequence”. These types of purification rely on a pretreatment modification step to generically modify all ribonucleic acids in a sample generically in such a way as to enable quantitative ribonucleic acid recovery from a tissue sample.

Reverse transcription PCR (qRT-PCR) is perhaps the most sensitive and flexible gene expression profiling method, which can be used to compare mRNA levels in different sample populations, in normal and diseased, e.g. tumor, tissues, with or without drug treatment, to characterize patterns of gene expression, to discriminate between closely related mRNAs, and to analyze RNA structure. As RNA cannot serve as a template for PCR, the first step in gene expression profiling by qRT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using gene specific primers, random hexamers, or oligo-dT primers, depending on the circumstances and the goal of expression profiling. For example, extracted RNA can be reverse-transcribed using a GeneAmp® RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ exonuclease activity but lacks a 3′-5′ proofreading endonuclease activity. Thus, TaqMan® PCR typically utilizes the 5′ exonuclease activity of Taq or Tth polymerase to hydrolyze a fluorescently-labelled hybridization probe bound to its target amplicon, but any enzyme with equivalent 5′ exonuclease activity can be used. Two oligonucleotide primers are used to generate an amplicon typical of a PCR reaction. A third oligonucleotide, or probe, is designed to hybridize to a nucleotide sequence located between the two PCR primers. The probe is non-extendible by Taq DNA polymerase enzyme, and is 5′ labeled with a reporter fluorescent dye and a 3′ labeled with a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second chromophore. One molecule of reporter dye is liberated for each new molecule synthesized, and detection of the unquenched reporter dye provides the basis for quantitative interpretation of the data.

qRT-PCR can be performed using commercially available equipment, such as, for example, ABI PRISM 7900™ Sequence Detection System™ (Perkin-Elmer-Applied Biosystems, Foster City, Calif., USA), or LightCycler® (Roche Molecular Biochemicals, Mannheim, Germany). In a preferred embodiment, the 5′ exonuclease procedure is run on a real-time quantitative PCR device such as the ABI PRISM 7900™ Sequence Detection System™ or one of the similar systems in this family of instruments. The system consists of a thermocycler, laser, charge-coupled device (CCD), camera and computer. The system amplifies samples in 96-well or 384 well formats on a thermocycler. During amplification, laser-induced fluorescent signal is collected in real-time through fiber optic cables for all reaction wells, and detected at the CCD. The system includes software for running the instrument and for analyzing the data. qRT-PCR assay data may initially be expressed as CT, or the threshold cycle, values. Fluorescence values are recorded during every PCR cycle and represent the amount of released fluorescent probe, which is directly proportional to product amplified to that point in the amplification reaction. The point when the fluorescent signal is first recorded as statistically significant is the threshold cycle (CT).

Methods are also known in the art for gene expression profiling without the need for an amplification step.

Given high-throughput sequencing technologies' low requirements of nucleotide sequence product, together with its deep coverage and base-scale resolution, its use has expanded to the field of transcriptomics. Transcriptomics is an area of research characterizing the RNA transcribed from a particular genome under investigation. Although transcriptomes are more dynamic than genomic DNA, these molecules provide direct access to gene regulation and protein information.

RNA sequencing (RNA-Seq) involves direct sequencing of complementary DNAs (cDNAs) using high-throughput DNA sequencing technologies followed by the mapping of the sequencing reads to the genome. This method involves the generation of a double-stranded cDNA library using random or oligo(dT) primers. The resulting library exhibits a bias towards the 5′ and 3′ ends of genes, which is useful for mapping the ends of genes and identifying transcribed regions. The cDNA is made from poly(A)+RNA, then fragmented by DNase I and ligated to adapters. These adapter-ligated cDNA fragments are then amplified and sequenced in a high-throughput manner to obtain short sequence reads. An Alternate Protocol describes the generation of a double-stranded cDNA library using random primers, but starting with poly(A)+RNA fragmented by partial hydrolysis. This provides a more uniform representation throughout the genes, which is helpful in quantifying exon levels, but is not as good for end mapping. Sequencing of the cDNA library may then be performed using any desired method known in the art, such as ion semiconductor sequencing, sequencing by ligation, sequencing by hybridization, polony sequencing, massively parallel signature sequencing, etc.

The nCounter® Gene Expression Assay (NanoString Technologies) provides methods and instruments for detecting the expression of up to 800 genes in a single reaction with high sensitivity and linearity across a broad range of expression levels. The nCounter assay is based on direct detection of mRNA molecules of interest using target-specific, color-coded probe pairs, referred to as “barcodes.” The methods do not require the conversion of mRNA to cDNA by reverse transcription or the amplification of the resulting cDNA by PCR. Each target gene of interest is detected using a pair of reporter and capture probes carrying 35- to 50-base target-specific sequences. Each reporter probe carries a unique color code at the 5′ end that enables the molecular barcoding of the genes of interest, while the capture probes all carry a biotin label at the 3′ end that provides a molecular handle for attachment of target genes to facilitate detection. After solution-phase hybridization between target mRNA and reporter-capture probe pairs, excess probes are removed and the probe/target complexes are aligned and immobilized in a disposable cartridge, which is then placed in the analyzer instrument for image acquisition and data processing. Hundreds of thousands of color codes designating mRNA targets of interest are directly imaged on the surface of the cartridge. The expression level of a gene is measured by counting the number of times the color-coded barcode for that gene is detected, and the barcode counts are then tabulated.

Direct RNA Sequencing (DRSTM) obtains a sequence from RNA molecules directly in a massively-parallel manner without RNA conversion to cDNA or other biasing sample manipulations such as ligation and amplification.

To be able to compare data from different tissue specimens, it may be necessary to correct for relative differences in input RNA quantity and quality. Such differences arise primarily from the variability inherent in processing surgical tissue specimens, including relative mass of tissue, the time between surgery and formalin fixation, and the storage time after fixation. Further variability might result from differences in the methods and/or reagents used for tissue fixation, and storage time following fixation. A further consideration is the cumulative variability accrued while processing each sample from RNA extraction through quantitation, reverse transcription to cDNA and PCR. This correction is accomplished by normalizing raw expression values relative to a set of genes that vary little in their median expression among different tissue specimens (“normalization reference genes”). It has been demonstrated that following the process of the present invention, including the normalization strategy used, RNA extracted from a variety of sources, using variety of fixative protocols and reagents can be analyzed successfully. RNAs frequently used to normalize patterns of gene expression include, among others, are mRNAs for glyceraldehyde-3-phosphate-dehydrogenase (GAPDH) and β-actin.

Gene expression profiling assays may be performed utilizing a microarray format. The term “microarray” refers to an ordered arrangement of hybridizable array elements, preferably polynucleotide probes, on a substrate. Microarray analysis for expression profiling involves typically the use of a series of nucleic acid probes arrayed in a one-, two- or even three-dimensional format on a surface, wherein each of the probes is has a unique location or address on the surface. The nucleic acid probes (including, for example but not limited to, oligonucleotides, PCR products, cDNA, plasmids or nucleic-acid-like synthetic polymers, all of which are capable of sequence specific hybridization) are made to be complementary to the sequences of the genes to be analyzed. The number of distinct probes that can be spotted to unique addressable locations on a single microarray is upwards of tens of thousands, and is constantly being revised upwards with improving state-of-the-art technology. The number of genes of interest to an investigator, and indeed the total number of expressed genes in any one RNA sample, is likely to be smaller than the probe capacity on a state-of-the art microarray; thus, the number of probes required for any one analysis is likely to be well within the upper limit of microarray probe capacity.

In a standard microarray protocol, the mRNA population is converted to cDNA by reverse transcription and globally labeled, e.g., with a fluorescent dye such as cy3 or cy5. The labeling step is either performed as part of the reverse transcription reaction, using a dye-labeled dNTP, or post reverse transcription using a chemically activated dye that couples to amino-dUTP incorporated during the reverse transcription step. The labeled product is then purified away from unincorporated dye and then placed on the array and the different gene sequences are allowed to hybridize to their complementary probes. The standard protocol requires a large amount of RNA, e.g. 100 (xg to 1 mg of total RNA, for starting material. In order to use less RNA starting material, the additional step of global amplification is frequently performed. Examples of global amplification methods include the SMART™ technology from BD Biosciences-Clontech (Palo Alto, Calif.), Ovation™ amplification technology from NuGEN™ Technologies, Inc. (San Carlos, Calif.), and RiboAmp™ RNA Amplification kit from Arcturus, Inc. (Mountain View, Calif.).

Therapy Based on Prognostic Score

Breast cancer is a heterogeneous disease comprising different subtypes defined on clinical, pathological, and molecular levels. In clinical practice, oncologists have recognized for years that the behavior of breast cancers is variable. Genomic assays can help to identify patients with early-stage ER+ breast cancers who do not need chemotherapy and are effectively treated with adjuvant endocrine agents alone. Alternatively, they can identify groups of patients with ER+ tumors who are more likely to be biologically homogenous and/or who might benefit from specific treatment strategies. Patients may be guided from chemotherapy+hormone therapy to hormone therapy alone based on a low recurrence probability; or guided to chemotherapy+hormone therapy based on a high recurrence probability.

Typically, the low-risk group is spared chemotherapy. In the case of a high probability of recurrent or metastatic disease, cytotoxic chemotherapy can include treatment with one or more of the following:

### Anthracyclines

Doxorubicin

Epirubicin

Liposomal doxorubicin

Mitoxantrone

### Taxanes

Paclitaxel

Docetaxel

Albumin-bound nanoparticle paclitaxel

Alkylating agents

Cyclophosphamide

### Fluoropyrimidines

Capecitabine

5-FU

### Antimetabolites

Methotrexate

Vinca alkaloids

Vinorelbine

Vinblastine

Vincristine

### Platinum

Carboplatin

Cisplatin

### Other

Gemcitabine

Mitomycin C

Eribulin mesylate

Combination regimens

CA: cyclophosphamide and doxorubicin

Docetaxel and doxorubicin

CAF: cyclophosphamide, doxorubicin, 5-fluorouracil

CMF: cyclophosphamide, methotrexate, 5-fluorouracil

Doxorubicin and paclitaxel

Docetaxel and capecitabine

Vinorelbine and epirubicin

Capecitabine and ixabepilone

### EXAMPLES

The following examples serve to illustrate the present invention. These examples are in no way intended to limit the scope of the invention.

### Example 1

### Measurement of Gene Expression in Archival Paraffin-Embedded Tissues

Archival breast tumor FPE blocks and matching frozen tumor sections were provided by Providence St. Joseph Medical Center, Burbank Calif. Excised tissues were incubated for five to ten hours in 10% neutral-buffered formalin before being alcohol-dehydrated and embedded in paraffin, following standard immunohistology procedures.

RNA was extracted from three 10 μmFPE (formalin fixed paraffin embedded) sections per each patient case. Paraffin was removed by xylene extraction followed by ethanol wash. RNA was isolated from sectioned tissue blocks using the protocol described in Example 3, with the exception that the MasterPure™ Purification kit (Epicentre, Madison, Wis.) was used for RNA extraction. In the cases of frozen tissue specimens, RNA was extracted using Trizol reagent according to the supplier's instructions (Invitrogen Life Technologies, Carlsbad, Calif.). Residual genomic DNA contamination was assayed by a TaqMan® quantitative PCR assay (no RT control) for (3-actin DNA. Samples with measurable residual genomic DNA were re-subjected to DNase I treatment, and assayed again for DNA contamination.

RNA was quantitated using the RiboGreen® fluorescence method (Molecular Probes, Eugene, Oreg.), and RNA size was analyzed by microcapillary electrophoresis using an Agilent 2100 Bioanalyzer (Agilent Technologies, Palo Alto, Calif.).

For each gene, the appropriate mRNA reference sequence (REFSEQ) accession number was identified and the consensus sequence accessed through the NCBI Entrez nucleotide database. qRT-PCR primers and probes were designed using Primer Express® (Applied Biosystems, Foster City, Calif.) and Primer3 programs. (Rosen and Skaletsky, Methods Mol. Biol. 132:365-386 (2000). Oligonucleotides were supplied by Biosearch Technologies Inc. (Novato, Calif.) and Integrated DNA Technologies (Coralville, Iowa). Amplicon sizes were preferably limited to less than 100 bases in length (see Results). Fluorogenic probes were dual-labeled with 5′-FAM as a reporter and 3′-BHQ-1 as a non-fluorogenic quencher.

Reverse transcription (RT) was carried out using a Superscript First-Strand Synthesis Kit for qRT-PCR (Invitrogen Corp., Carlsbad, Calif.). Total FPE RNA and pooled gene specific primers were present at 10-50 ng/wl and 100 nM (each) respectively.

TaqMan reactions were performed in 384 well plates according to instructions of the manufacturer, using Applied Biosystems Prism® 7900HT TaqMan instruments. Expression of each gene was measured either in duplicate 5 μL reactions using cDNA synthesized from 1 ng of total RNA per reaction well, or in single reactions using cDNA synthesized from 2 ng of total RNA, as indicated. Final primer and probe concentrations were 0.9 μM (each primer) and 0.2 μM, respectively. PCR cycling was carried out as follows: 95° C. 10 minutes for one cycle, 95° C. 20 seconds, and 60° C. 45 seconds for 40 cycles. To verify that the qRT-PCR signals derived from RNA rather than genomic DNA, for each gene tested a control identical to the test assay but omitting the RT reaction (no RT control) was included. The threshold cycle for a given amplification curve during qRT-PCR occurs at the point the fluorescent signal from probe cleavage grows beyond a specified fluorescence threshold setting. Test samples with greater initial template exceed the threshold value at earlier amplification cycle numbers than those with lower initial template quantities.

### Example 2

### Statistical Analysis

Statistical analyses were performed using MedCalc Version 11.5.1.0 for Windows 7 (MedCalc Software, Gent, Belgium).

Heat Map: A hierarchical cluster analysis using Euclidian distances (unsupervised in the acyltransferase dimension) was performed on the Agilent expression values of all acyltransferase probes that remained after probe verification in order to construct a heat map of all breast cancer patients.

Correlation to patient outcome: In order to identify candidate DHHC proteins for roles in tumorigenesis and patient outcomes, student T-tests were performed on all acyltransferases against various clinical variables to compare normal breast tissue with tumour breast tissue. Receiver operating characteristic (ROC) curves using patient death as a classification variable were performed in order to determine the optimal cut-off value for each probe expression values. Patients were then individually classified as over- or under-expressors for each acyltransferase. Cox proportional-hazard regression tests were performed for Univariate analysis using an enter model for both survival and recurrence-free survival in order to further identify DHHC proteins that correlate with patient outcome. Multivariate analysis was then performed on the acyltransferases that showed significance in the Univariate analysis. This was performed using a Backward enter model with variables removed if P>0.10. The log rank test on Kaplan-Meier survival curves were plotted for survival and recurrence free survival.

### Example 3

### Results

To establish a global fatty acyl transferase expression pattern in breast cancers, we mined a breast cancer database to extract the data corresponding to the expression of all 27 protein fatty acyltransferases. The data set comprised genome-wide analysis of the transcriptomes obtained from the RNA isolated from the primary tumors of 167 women with untreated invasive breast cancer, and 10 “normal” reduction mammoplasty specimens. This molecularly defined cohort includes complete baseline clinicopathologic data, received guideline-based local and systemic adjuvant therapy, lifelong follow-up for each patient. 50% of the cohort has experienced cancer relapse. The microarray data was paired with cDNA of each tumour, and a formalin-fixed tissue microarray containing tumour plugs of each patient for ImmunoHistoChemistry (IHC) staining.

It was determined that zDHHC-5, -9 and -20 were overexpressed (at greater than 5 times the standard deviation) in the vast majority of tumours, while the expressions of zDHHC1, and of zDHHC2, a postulated tumour suppressor originally named REAM (Reduced Expression Associated with Metastasis) were reduced in a large number of tumours. It was also determined that that the high expression of a number of zDHHC-PATs (zDHHC-5, -7, -9, -14, -20, -23 and -24) within the patient population correlated with either low patient survival or tumour recurrence or both. High expression of zDHHC3 and the membrane-bound O-acyl transferase HHAT correlated with extremely high patient survival.

The combinatorial high expression of zDHHC-7, -14 and -20 had an additive effect with regards to the rate of tumour recurrence and death, showing a combined health hazard ratio of 9.32 (HR=9.32) and no patient surviving 4.5 years past diagnostic date while patients with normal levels of zDHHC-7, -14 and -20 showed a 85% ten year survival rate. Importantly. ˜11% of patients were “triple positive” for the overexpression of zDHHC-7, -14, -20.

Additionally, multivariate analysis (a statistical method to determine in this case which prognostic factors outperformed the others) demonstrated that the prognostic ability of the combination of these three biomarkers outperformed those of tumour stage, tumour grade and Progesterone Receptor (PR) expression level: three current gold standards for providing breast cancer patient prognosis

### Example 4

### Validation Study

A validation of the prognostic value of the zDHHC7, zDHHC14 and zDHHC20 three biomarker panel was carried out on a cohort of 997 patients comprising the discovery dataset from the METABRIC (Curtis et al., Nature 2012, doi:10.1038/nature10983) study carried out at the British Columbia Cancer Research Centre (BCCRC).

Protein expression in the METABRIC validation study was assessed using the Illumina HT-12 v3 platform (Illumina_Human_WG-v3), and a heat map calculated. Additional gene targets for the genes associated with breast cancer subtype classification were also identified: (ER (ESR1), PR (PGR), Her2 (ERBB2), Ki67 (MKI67), EGFR, CK5/6 (KRT5), Aurora kinase A (AURKA), and survivin (EPR-1 and BIRC5)).

Biomarker identity and binarization cut point information was provided from the data described in Example 3 via ROC by time analysis of survival data. The percentile cut points obtained from that study (75^{th}, 70^{th} and 65^{th} percentiles respectively for zDHHC7, zDHHC14 and zDHHC20) were used in this validation exercise to avoid the issue of selecting cut points specifically for the METABRIC data.

Kaplan-Meier analysis and Cox proportional hazards model fit assessment for overall survival was carried out to evaluate the prognostic value of the biomarker panel. The three biomarker panel consisting of expression values for gene targets zDHHC7, zDHHC14 and zDHHC20 showed prognostic value in a multivariable Cox model (p=0.0015). The panel showed independent prognostic value in a multivariable Cox model also containing PAM50 breast cancer subtypes (p=0.022), in a multivariable Cox model containing clinical covariates age, grade, tumour size and nodal status (p=0.0016), and in a multivariable Cox model containing both breast cancer subtypes and clinical covariates (p=0.010). Hazard ratio estimates from the validation study cohort tended to be smaller than observed in the original study, as would be expected due to issues of model overfitting in original data series, though as noted above, the panel continued to show prognostic value in an independent data cohort.

Binarization of each gene target yielded the following case counts:

> with(sdf, table(zDHHC7_c75))
zDHHC7_c75

| zDHHC7_Neg | zDHHC7_Pos |
|---|---|
| 737 | 246 |

> with(sdf, table(zDHHC7_c75, BrCaf))
BrCaf

| zDHHC7_c75 | LumA | LumB | Her2 | Basal | Normal |
|---|---|---|---|---|---|
| zDHHC7_Neg | 357 | 209 | 56 | 79 | 35 |
| zDHHC7_Pos | 74 | 78 | 38 | 39 | 14 |

> with(sdf, table(zDHHC14_c70))
zDHHC14_c70

| zDHHC14_Neg | zDHHC14_Pos |
|---|---|
| 688 | 295 |

> with(sdf, table(zDHHC14_c70, BrCaf))
BrCaf

| zDHHC14_c70 | LumA | LumB | Her2 | Basal | Normal |
|---|---|---|---|---|---|
| zDHHC14_Neg | 329 | 235 | 45 | 51 | 25 |
| zDHHC14_Pos | 102 | 52 | 49 | 67 | 24 |

> with(sdf, table(zDHHC20_c65))
zDHHC20_c65

| zDHHC20_Neg | zDHHC20_Pos |
|---|---|
| 639 | 344 |

> with(sdf, table(zDHHC20_c65, BrCaf))
BrCaf

| zDHHC20_c65 | LumA | LumB | Her2 | Basal | Normal |
|---|---|---|---|---|---|
| zDHHC20_Neg | 334 | 181 | 39 | 44 | 40 |
| zDHHC20_Pos | 97 | 106 | 55 | 74 | 9 |

Combining across all three binarized scores yielded the following case counts

> with(sdf, table(Sum71420))
Sum71420

| None | One only | Two only | All three |
|---|---|---|---|
| 358 | 398 | 194 | 33 |

> with(sdf, table(Sum71420, BrCaf))
BrCaf

| Sum71420 | LumA | LumB | Her2 | Basal | Normal |
|---|---|---|---|---|---|
| None | 211 | 103 | 11 | 19 | 14 |
| One only | 169 | 135 | 36 | 32 | 25 |
| Two only | 49 | 46 | 35 | 53 | 8 |
| All three | 2 | 3 | 12 | 14 | 2 |

Case counts for positivity on all three biomarkers was too small to obtain survival analysis estimates within breast cancer subtypes (PAM50 subtypes) so the categories “Two only” and “All three” were combined:

> with(sdf, table(Sumg71420))
Sumg71420

| None | One only | Two or three |
|---|---|---|
| 358 | 398 | 227 |

> with(sdf, table(Sumg71420, BrCaf))
BrCaf

| Sumg71420 | LumA | LumB | Her2 | Basal | Normal |
|---|---|---|---|---|---|
| None | 211 | 103 | 11 | 19 | 14 |
| One only | 169 | 135 | 36 | 32 | 25 |
| Two or three | 51 | 49 | 47 | 67 | 10 |

Prognostic Value Assessment:

Biomarkers Alone:

In a multivariable Cox model of overall survival containing binarized variables for zDHHC7, zDHHC14 and zDHHC20, the three marker panel showed prognostic value (likelihood ratio chi-square (3 d.f.)=15.4, p=0.0015).

> bmfml <− coxph(Surv(survyrs, survstat)~zDHHC7_c75 + zDHHC14_c70 +
zDHHC20_c65, data = sdf)
> bmrm1 <− coxph(Surv(survyrs, survstat)~1, data = sdf)
> summary(bmfm1)
Call: coxph(formula = Surv(survyrs, survstat)~zDHHC7_c75 + zDHHC14_c70 +
zDHHC20_c65, data = sdf)
n = 983, number of events = 446

| | coef | exp(coef) | se(coef) | z | Pr(>\|z\|) | |
|---|---|---|---|---|---|---|
| zDHHC7_c75zDHHC7_Pos | 0.004399 | 1.004408 | 0.110506 | 0.040 | 0.96825 | |
| zDHHC14_c70zDHHC14_Pos | 0.225903 | 1.253455 | 0.101315 | 2.230 | 0.02577 | * |
| zDHHC20_c65zDHHC20_Pos | 0.312090 | 1.366278 | 0.098342 | 3.174 | 0.00151 | ** |

Signif. codes: 0 ‘***’ 0.001 ‘**’ 0.01 ‘*’ 0.05 ‘.’ 0.1 ‘’ 1

| | exp(coef) | exp(−coef) | lower .95 | upper .95 |
|---|---|---|---|---|
| zDHHC7_c75zDHHC7_Pos | 1.004 | 0.9956 | 0.8088 | 1.247 |
| zDHHC14_c70zDHHC14_Pos | 1.253 | 0.7978 | 1.0277 | 1.529 |
| zDHHC20_c65zDHHC20_Pos | 1.366 | 0.7319 | 1.1268 | 1.657 |

Concordance = 0.568 (se = 0.014)
Rsquare = 0.016 (max possible = 0.997)

| | | | |
|---|---|---|---|
| Likelihood ratio test = | 15.39 | on 3 df, | p = 0.001511 |
| Wald test = | 15.85 | on 3 df, | p = 0.001217 |
| Score (logrank) test = | 15.97 | on 3 df, | p = 0.00115 |

Biomarkers Plus Breast Cancer Subtypes:

In a multivariable Cox model of overall survival containing binarized variables for zDHHC7, zDHHC14 and zDHHC20 and breast cancer subtypes, the three marker panel showed independent prognostic value (likelihood ratio chi-square (3 d.f.)=9.65, p=0.022).

> bmfm2 <− coxph(Surv(survyrs, survstat)~zDHHC7_c75 + zDHHC14_c70 +
zDHHC20_c65 + BrCaf, data = sdf)
> bmrm2 <− coxph(Surv(survyrs, survstat)~BrCaf, data = sdf)
> summary(bmfm2)
Call: coxph(formula = Surv(survyrs, survstat)~zDHHC7_c75 + zDHHC14_c70 +
zDHHC20_c65 + BrCaf, data = sdf)
n = 979, number of events = 445
(4 observations deleted due to missingness)

| | coef | exp(coef) | se(coef) | z | Pr(>\|z\|) | |
|---|---|---|---|---|---|---|
| zDHHC7_c75zDHHC7_Pos | −0.05165 | 0.94966 | 0.11313 | −0.457 | 0.64800 | |
| zDHHC14_c70zDHHC14_Pos | 0.21697 | 1.24231 | 0.10824 | 2.005 | 0.04501 | * |
| zDHHC20_c65zDHHC20_Pos | 0.24827 | 1.28181 | 0.10322 | 2.405 | 0.01616 | * |
| BrCafLumB | 0.36053 | 1.43409 | 0.11463 | 3.145 | 0.00166 | ** |
| BrCafHer2 | 0.47971 | 1.61560 | 0.16220 | 2.957 | 0.00310 | ** |
| BrCafBasal | 0.18720 | 1.20587 | 0.16781 | 1.116 | 0.26462 | |
| BrCafNormal | 0.16255 | 1.17651 | 0.25179 | 0.646 | 0.51854 | |

Signif. codes: 0 ‘***’ 0.001 ‘**’ 0.01 ‘*’ 0.05 ‘.’ 0.1 ‘’ 1

| | exp(coef) | exp(−coef) | lower .95 | upper .95 |
|---|---|---|---|---|
| zDHHC7_c75zDHHC7_Pos | 0.9497 | 1.0530 | 0.7608 | 1.185 |
| zDHHC14_c70zDHHC14_Pos | 1.2423 | 0.8050 | 1.0048 | 1.536 |
| zDHHC20_c65zDHHC20_Pos | 1.2818 | 0.7801 | 1.0470 | 1.569 |
| BrCafLumB | 1.4341 | 0.6973 | 1.1455 | 1.795 |
| BrCafHer2 | 1.6156 | 0.6190 | 1.1756 | 2.220 |
| BrCafBasal | 1.2059 | 0.8293 | 0.8679 | 1.675 |
| BrCafNormal | 1.1765 | 0.8500 | 0.7182 | 1.927 |

Concordance = 0.602 (se = 0.015)
Rsquare = 0.03 (max possible = 0.997)

| | | | |
|---|---|---|---|
| Likelihood ratio test = | 30.1 | on 7 df, | p = 9.087e−05 |
| Wald test = | 30.69 | on 7 df, | p = 7.079e−05 |
| Score (logrank) test = | 31.18 | on 7 df, | p = 5.756e−05 |

> anova(bmrm2, bmfm2)
Analysis of Deviance Table
Cox model: response is Surv(survyrs, survstat)
Model 1: ~BrCaf
Model 2: ~zDHHC7_c75 + zDHHC14_c70 + zDHHC20_c65 + BrCaf

| | loglik | Chisq | Df | P(>\|Chi\|) | |
|---|---|---|---|---|---|
| 1 | −2760.7 | | | | |
| 2 | −2755.8 | 9.6491 | 3 | 0.0218 | * |

Signif. codes: 0 ‘***’ 0.001 ‘**’ 0.01 ‘*’ 0.05 ‘.’ 0.1 ‘’ 1

Biomarkers Plus Clinical Covariates:

In a multivariable Cox model of overall survival containing binarized variables for zDHHC7, zDHHC14 and zDHHC20 and the standard breast cancer regression model clinical covariates age, grade, tumour size and nodal status, the three marker panel showed independent prognostic value (likelihood ratio chi-square (3 d.f.)=15.2, p=0.0016).

> bmfm3 <− coxph(Surv(survyrs, survstat)~zDHHC7_c75 + zDHHC14_c70 +
zDHHC20_c65 ++ age + grade + tsize + nodestat, data = sdf)
> bmrm3 <− coxph(Surv(survyrs, survstat)~age + grade + tsize +
nodestat, data = sdf)
> summary (bmfm3)
Call: coxph(formula = Surv(survyrs, survstat)~zDHHC7_c75 + zDHHC14_c70 +
zDHHC20_c65 + age + grade + tsize + nodestat, data = sdf)
n = 967, number of events = 433
(16 observations deleted due to missingness)

| | coef | exp(coef) | se(coef) | z | Pr(>\|z\|) | |
|---|---|---|---|---|---|---|
| zDHHC7_c75zDHHC7_Pos | −0.02532 | 0.97500 | 0.11454 | −0.221 | 0.825073 | |
| zDHHC14_c70zDHHC14_Pos | 0.23845 | 1.26928 | 0.10507 | 2.269 | 0.023246 | * |
| zDHHC20_c65zDHHC20_Pos | 0.31139 | 1.36532 | 0.10188 | 3.057 | 0.002239 | ** |
| age(50, 65] | 0.17188 | 1.18754 | 0.14385 | 1.195 | 0.232132 | |
| age(65, 100] | 0.70794 | 2.02981 | 0.13495 | 5.246 | 1.55e−07 | *** |
| gradegrade3 | 0.26810 | 1.30748 | 0.10317 | 2.599 | 0.009356 | ** |
| tsize2-5 cm | 0.26178 | 1.29924 | 0.10778 | 2.429 | 0.015150 | * |
| tsize >5 cm | 0.71440 | 2.04297 | 0.21571 | 3.312 | 0.000927 | *** |
| nodestatNodePos | 0.57838 | 1.78315 | 0.10380 | 5.572 | 2.51e−08 | *** |

Signif. codes: 0 ‘***’ 0.001 ‘**’ 0.01 ‘*’ 0.05 ‘.’ 0.1 ‘’ 1

| | exp(coef) | exp(−coef) | lower .95 | upper .95 |
|---|---|---|---|---|
| zDHHC7_c75zDHHC7_Pos | 0.975 | 1.0256 | 0.7790 | 1.220 |
| zDHHC14_c70zDHHC14_Pos | 1.269 | 0.7879 | 1.0330 | 1.560 |
| zDHHC20_c65zDHHC20_Pos | 1.365 | 0.7324 | 1.1182 | 1.667 |
| age(50, 65] | 1.188 | 0.8421 | 0.8958 | 1.574 |
| age(65, 100] | 2.030 | 0.4927 | 1.5581 | 2.644 |
| gradegrade3 | 1.307 | 0.7648 | 1.0681 | 1.600 |
| tsize2-5 cm | 1.299 | 0.7697 | 1.0518 | 1.605 |
| tsize >5 cm | 2.043 | 0.4895 | 1.3386 | 3.118 |
| nodestatNodePos | 1.783 | 0.5608 | 1.4549 | 2.185 |

Concordance = 0.66 (se = 0.015)
Rsquare = 0.122 (max possible = 0.996)

| | | | |
|---|---|---|---|
| Likelihood ratio test = | 125.5 | on 9 df, | p = 0 |
| Wald test = | 122.9 | on 9 df, | p = 0 |
| Score (logrank) test = | 126.7 | on 9 df, | p = 0 |

> anova(bmrm3, bmfm3)
Analysis of Deviance Table
Cox model: response is Surv(survyrs, survstat)
Model 1: ~age + grade + tsize + nodestat
Model 2: ~zDHHC7_c75 + zDHHC14_c70 + zDHHC20_c65 + age + grade + tsize + nodestat

| | loglik | Chisq | Df | P(>\|Chi\|) | |
|---|---|---|---|---|---|
| 1 | −2641.1 | | | | |
| 2 | −2633.5 | 15.206 | 3 | 0.001649 | ** |

Signif. codes: 0 ‘***’ 0.001 ‘**’ 0.01 ‘*’ 0.05 ‘.’ 0.1 ‘’ 1

Biomarkers Plus Clinical Covariates and Breast Cancer Subtypes:

In a multivariable Cox model of overall survival containing binarized variables for zDHHC7, zDHHC14 and zDHHC20 and the standard breast cancer regression model clinical covariates age, grade, tumour size and nodal status, plus breast cancer subtypes, the three marker panel showed independent prognostic value (likelihood ratio chi-square (3 d.f.)=11.3, p=0.010).

> bmfm4 <− coxph(Surv(survyrs, survstat)~zDHHC7_c75 + zDHHC14_c70 +
zDHHC20_c65 ++ age + grade + tsize + nodestat + BrCaf, data = sdf)
> bmrm4 <− coxph(Surv(survyrs, survstat)~age + grade + tsize +
nodestat + BrCaf, data = sdf)
> summary(bmfm4)
Call: coxph(formula = Surv(survyrs, survstat)~zDHHC7_c75 + zDHHC14_c70 +
zDHHC20_c65 + age + grade + tsize + nodestat + BrCaf, data = sdf)
n = 963, number of events = 432
(20 observations deleted due to missingness)

| | coef | exp(coef) | se(coef) | z | Pr(>\|z\|) | |
|---|---|---|---|---|---|---|
| zDHHC7_c75zDHHC7_Pos | −0.05864 | 0.94305 | 0.11637 | −0.504 | 0.61433 | |
| zDHHC14_c70zDHHC14_Pos | 0.20735 | 1.23041 | 0.11018 | 1.882 | 0.05983 | . |
| zDHHC20_c65zDHHC20_Pos | 0.28636 | 1.33157 | 0.10567 | 2.710 | 0.00673 | ** |
| age(50, 65] | 0.17075 | 1.18619 | 0.14774 | 1.156 | 0.24779 | |
| age(65, 100] | 0.71202 | 2.03810 | 0.14303 | 4.978 | 6.42e−07 | *** |
| gradegrade3 | 0.20391 | 1.22618 | 0.11169 | 1.826 | 0.06791 | . |
| tsize2-5 cm | 0.26491 | 1.30331 | 0.10851 | 2.441 | 0.01464 | * |
| tsize >5 cm | 0.68870 | 1.99113 | 0.21720 | 3.171 | 0.00152 | ** |
| nodestatNodePos | 0.56323 | 1.75634 | 0.10504 | 5.362 | 8.22e−08 | *** |
| BrCafLumB | 0.14966 | 1.16144 | 0.12135 | 1.233 | 0.21746 | |
| BrCafHer2 | 0.33587 | 1.39916 | 0.17394 | 1.931 | 0.05349 | . |
| BrCafBasal | 0.13964 | 1.14986 | 0.18547 | 0.753 | 0.45151 | |
| BrCafNormal | 0.30982 | 1.36318 | 0.26337 | 1.176 | 0.23945 | |

Signif. codes: 0 ‘***’ 0.001 ‘**’ 0.01 ‘*’ 0.05 ‘.’ 0.1 ‘’ 1

| | exp(coef) | exp(−coef) | lower .95 | upper .95 |
|---|---|---|---|---|
| zDHHC7_c75zDHHC7_Pos | 0.943 | 1.0604 | 0.7507 | 1.185 |
| zDHHC14_c70zDHHC14_Pos | 1.230 | 0.8127 | 0.9914 | 1.527 |
| zDHHC20_c65zDHHC20_Pos | 1.332 | 0.7510 | 1.0825 | 1.638 |
| age(50, 65] | 1.186 | 0.8430 | 0.8880 | 1.585 |
| age(65, 100] | 2.038 | 0.4907 | 1.5399 | 2.698 |
| gradegrade3 | 1.226 | 0.8155 | 0.9851 | 1.526 |
| tsize2-5 cm | 1.303 | 0.7673 | 1.0536 | 1.612 |
| tsize >5 cm | 1.991 | 0.5022 | 1.3008 | 3.048 |
| nodestatNodePos | 1.756 | 0.5694 | 1.4296 | 2.158 |
| BrCafLumB | 1.161 | 0.8610 | 0.9156 | 1.473 |
| BrCafHer2 | 1.399 | 0.7147 | 0.9950 | 1.968 |
| BrCafBasal | 1.150 | 0.8697 | 0.7994 | 1.654 |
| BrCafNormal | 1.363 | 0.7336 | 0.8135 | 2.284 |

Concordance = 0.666 (se = 0.015)
Rsquare = 0.125 (max possible = 0.996)

| | | | |
|---|---|---|---|
| Likelihood ratio test = | 128.9 | on 13 df, | p = 0 |
| Wald test = | 126 | on 13 df, | p = 0 |
| Score (logrank) test = | 130.2 | on 13 df, | p = 0 |

> anova(bmrm4, bmfm4)
Analysis of Deviance Table
Cox model: response is Surv(survyrs, survstat)
Model 1: ~age + grade + tsize + nodestat + BrCaf
Model 2: ~zDHHC7_c75 + zDHHC14_c70 + zDHHC20_c65 + age + grade + tsize + nodestat + BrCaf

| | loglik | Chisq | Df | P(>\|Chi\|) | |
|---|---|---|---|---|---|
| 1 | −2628.7 | | | | |
| 2 | −2623.0 | 11.269 | 3 | 0.01036 | * |

Signif. codes: 0 ‘***’ 0.001 ‘**’ 0.01 ‘*’ 0.05 ‘.’ 0.1 ‘’ 1

### Example 5

The Alberta tumour bank data set comprised genome-wide analysis of the transcriptomes obtained from the RNA isolated from the primary tumors of 167 untreated non-metastatic invasive breast cancer, and 10 “normal” reduction mammoplasty specimens. This molecularly defined cohort serves as an excellent dataset for the discovery of prognostically important biomarkers, as each patient has complete baseline clinicopathologic data, received curative-intent guideline-based local and systemic adjuvant therapy and on-going follow-up. The microarray data are analyzed from patient tissues that are paired with protein extracts, formalin-fixed tissues and tumour plugs.

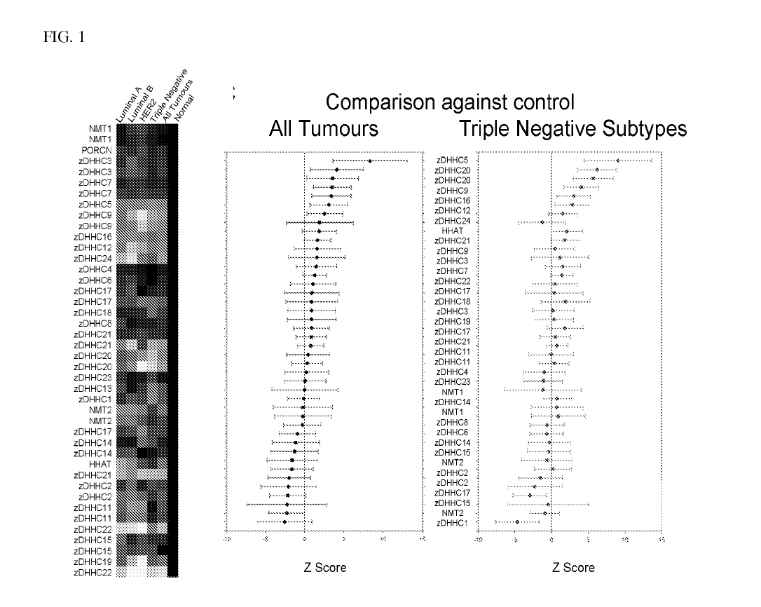

FIG. 1 depicts a “heat map” showing the averaged expression (log₂ transformed) of various PFATs per tumor subtype, and the average expression of each PFAT plotted as a Z score distance from the mean of the normal breast tissue. Error bars are the propagation of the standard deviation of the mean. In some cases, results correspond to more than one oligonucleotide sequence present on the micro-array (e.g. NMT1).

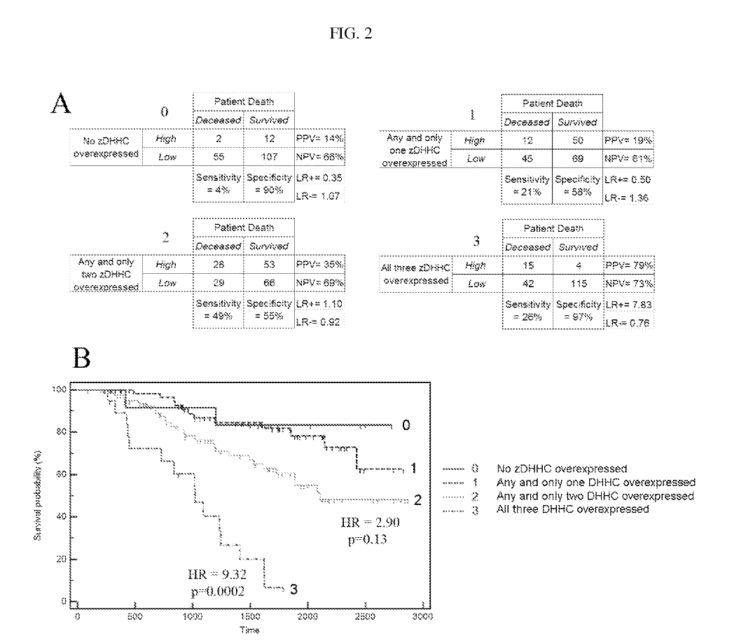

FIG. 2 depicts a combinatorial prognostic analysis of zDHHC-PATs-7, -14 and -20 expression. In panel A, Fisher's exact tests of the various combinations of zDHHC-PATs-7, -14 and -20 expression levels are depicted. In panel B, Kaplan Meier survival curves of various combinations of zDHHC-PATs-7, -14 and -20 expression levels are depicted. Abbreviations: PPV positive predictive value, NPV negative predictive value, LR+ positive likelihood ratio, LR− negative likelihood ratio, HR hazard ratio.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

The use of “or” herein means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. All patents and publications mentioned in the specification are indicative of the levels of those of ordinary skill in the art to which the invention pertains prior to the filing date of the disclosure. Nothing herein is to be construed as an admission that the inventors are not entitled to antedate such disclosure by virtue of prior disclosure.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A method of determining a prognostic classification for a patient diagnosed with breast cancer, comprising:
obtaining an RNA or protein sample from a tumor cell sample obtained from the patient;
determining gene expression levels for a plurality of protein fatty acyl transferase genes by
(i) contacting mRNA sequences present in the RNA sample expressed by the plurality of protein fatty acyl transferase genes, or nucleic acid sequences prepared therefrom, with a detectable label which binds thereto and provides a sequence-specific signal therefrom, and
generating one or more assay results indicative of the gene expression of the plurality of fatty acyl transferase genes from the detectable label, or
(ii) contacting the protein sample with a plurality of reagents which specifically bind for detection proteins expressed from the plurality of protein fatty acyl transferase genes, and
generating one or more assay results indicative of the gene expression of the plurality of protein fatty acyl transferase genes from protein bound to the plurality of reagents;
calculating the prognostic classification by correlating the assay results to a likelihood of an outcome result for the patient.

2. A method according to claim 1, wherein the likelihood of an outcome result is a likelihood that the patient will respond to a selected treatment regimen.

3. A method according to claim 1, wherein the likelihood of an outcome result is a likelihood of tumor recurrence.

4. A method according to claim 1, wherein the likelihood of an outcome result is a likelihood of metastatic disease.

5. A method according to claim 1, wherein the likelihood of an outcome result is a likelihood of mortality.

6. A method according to claim 1, wherein the one or more assay results comprise a plurality of individual assay results indicative of the gene expression level of each protein fatty acyl transferase gene in the plurality of protein fatty acyl transferase genes.

7. A method according to claim 6, wherein the likelihood of an outcome result is expressed as a single value that is a function of the plurality of individual assay results.

8. A method according to claim 7, wherein the likelihood of an outcome result is expressed as an odds ratio.

9. A method according to claim 7, wherein the likelihood of an outcome result is expressed as a hazard ratio.

10. A method according to claim 1, wherein the plurality of protein fatty acyl transferase genes comprise one or more genes selected from the group consisting of NMT1, NMT2, Hhat, Porc, zDHHC-3, zDHHC-5, zDHHC-7, zDHHC-9, zDHHC-14, zDHHC-20, zDHHC-23 and zDHHC-24.

11. A method according to claim 1, wherein the plurality of protein fatty acyl transferase genes comprise two or more of zDHHC-5, zDHHC-7, zDHHC-9, zDHHC-14 and zDHHC-20.

12. A method according to claim 10, wherein the plurality of protein fatty acyl transferase genes comprise each of zDHHC-7, zDHHC-14 and zDHHC-20.

13. A method according to claim 1, wherein the prognostic classification further comprises correlating one or more assay results indicative of the expression of one or more biomarkers selected from the group consisting of H-Ras, N-Ras, estrogen receptor, progesterone receptor, HER1, HER2, and cytokeratin 5/6, to the likelihood of an outcome result for the patient.

14. A method according to claim 1, wherein the one or more assay results indicative of the gene expression of the plurality of protein fatty acyl transferases genes are generated by direct detection of mRNA molecules by hybridization to a target-specific color-coded nucleic acid probe corresponding to each of the protein fatty acyl transferases genes of interest, and the step of generating one or more assay results indicative of the gene expression of the plurality of fatty acyl transferases genes comprises detection of hybridization of each target-specific color-coded nucleic acid probe to its corresponding mRNA.

15. A method according to claim 14, wherein the method further comprises hybridization of a nucleic acid capture probe to each of the protein fatty acyl transferases genes of interest, the nucleic acid capture probe comprising a first member of a binding pair, and capturing the first member of the binding pair with a cognate second member of the binding pair, the second member of the binding pair being immobilized to a surface.

16. A method according to claim 1, wherein the one or more assay results indicative of the gene expression of the plurality of protein fatty acyl transferases genes are generated by preparing a set of amplified nucleic acids by enzymatically amplifying mRNA sequences present in the RNA sample using a primer-dependent nucleic acid polymerase, wherein primers used to prepare the set of amplified nucleic acids are selected to amplify mRNA expressed by the plurality of protein fatty acyl transferase genes, and
generating the one or more assay results indicative of the gene expression of the plurality of fatty acyl transferases genes from the set of amplified nucleic acids.

17. A method according to claim 16, wherein the one or more assay results are generated by contacting using a hybridization array comprising immobilized nucleic acids selected to hybridize to sequences amplified from mRNA expressed by the plurality of protein fatty acyl transferase genes.

18. A method according to claim 16, wherein the one or more assay results are generated by quantitative reverse transcription-polymerase chain reaction (qRT-PCR).

19. A method according to claim 1, wherein the one or more assay results indicative of the gene expression of the plurality of protein fatty acyl transferases genes are generated by immunoassay detection of proteins expressed from the plurality of fatty acyl transferase genes.

20. A method according to claim 19, wherein the immunoassay detection is performed by antibody microarray, Western blot, SELDI-TOF-MS, fluorescent immunoassay, ELISA, or flow cytometry.

21. A method according to claim 1, wherein the prognostic classification further comprises combining the one or more assay results indicative of the gene expression of one or more of the plurality of fatty acyl transferase genes with one or more gene expression profiles indicative of breast cancer prognosis to determine the likelihood of an outcome result for the patient.

22. A method according to claim 21, wherein the one or more gene expression profiles indicative of breast cancer prognosis comprise one or more of a PAM50 risk of recurrence score, an OncotypeDX recurrence score, a Breast Cancer Index score, a DCIS Score, and an IHC4 recurrence risk score.

23. A method according to claim 1, further comprising selecting a treatment regimen based on the prognostic classification.
